(19) United States
(12) Patent Application Publication
(10) Pub. No.: US 2013/0041302 A1
(43) Pub. Date: Feb. 14, 2013

(54) FUNCTIONAL EXERCISE GLOVE AND 19+19 DEGREE ERGONOMIC BRACING DEVICES

(71) Applicant: Charlton Hudson Williams, Panama City Beach, FL (US)

(72) Inventor: Charlton Hudson Williams, Panama City Beach, FL (US)

(73) Assignee: NATRAFLEX, INC., Panama City Beach, FL (US)

(21) Appl. No.: 13/571,200

(22) Filed: Aug. 9, 2012

Related U.S. Application Data
(60) Provisional application No. 61/574,756, filed on Aug. 9, 2011.

(51) Int. Cl. A61F 5/01; A41D 19/015

(52) U.S. Cl. 602/22; 2/159; 602/21

(57) ABSTRACT

A functional exercise glove includes a dorsal-side layer having in-plane resistance to stretch in the longitudinal direction of the glove that is greater than its in-plane resistance to stretch transverse to the longitudinal direction. At least one fingertip member transfers an extension/flexion force from a user's finger to the dorsal-side layer upon flexion of the finger. The glove is configured to react the extension/flexion force from the dorsal-side layer into at least one of the heel of the user's hand or the user's wrist. The dorsal-side layer resists extension from flexion of the fingers thereby working finger, hand, wrist, forearm muscles, tendons, ligaments, bones, and nerves specific to natural movement patterns. The exercise glove can be used during a job function, regular activity, and while playing a sport like golf, tennis, etc.

### CROSS-REFERENCES TO RELATED APPLICATIONS

This application claims the benefit of U.S. Provisional Application No. 61/574,756, filed Aug. 9, 2011, the entire disclosure of which is hereby incorporated herein by reference.

### FIELD OF THE INVENTION

This application discloses functional exercise gloves and ergonomic braces for exercising and bracing the fingers, hands, wrist and forearm of a user.

### BACKGROUND

Biomechanical Issues of the Fingers, Hand, Wrist and Forearm

A human hand includes many moving parts and is capable of a tremendous number of movements. Additional movement of the hand results from articulation of the wrist and/or the radioulnar (elbow) joint. When hand positions/motions are combined with those of the wrist and radioulnar joint, the manipulations performed by the human hand are virtually infinite in number. The hand can shape itself to almost any object that can be grasped primarily due to the large number of joints within the hand as well as the articular contours, numerous muscles, and significant innervations. The musculoskeletal structure of the hand provides both significant range of motion (ROM) as well as high rigidity. The rigidity is produced in part by the tight fitting joints between the second and third metacarpals and the trapezoid and capitate bones. The fourth and fifth metacarpals allow for ROM of less than 30 degrees relative to the hamate bone. The ROM about the metacarpal (MCP) tri-axial joints is significantly greater than those possible in the carpometacarpal (CMC) joints. The sagittal plane ROM is typically in the range of 70 to 95 degrees of flexion (index finger to little finger/respectively). Extension tends to vary from person to person around the MCP joints. The proximal interphalangeal (PIP) joints and distal interphalangeal (DIP) joints of the four digits allow sagittal plane motions only. The PIP joint typically allows approximately 110 degrees of flexion, while the DIP allows approximately 90 degrees of flexion. Hyperextension of these joints is possible but is very limited compared to flexion ROM.

The thumb can move in two planes, the sagittal and frontal, thereby allowing its movement to trace a cone. The principal motions of the hand include abduction and adduction, with flexion and extension occurring about the MCP and IP joints. The primary role of the thumb is to provide opposition to the fingers. Flexion ROM around the MCP joint is as much as 90 degrees but may be considerably less. Extension of the joint may range up to 15 degrees normally.

The muscular and ligamentous structures of the hand are quite extensive and complex and provide the stability and control necessary for grasping heavy objects with varied shapes as well as the mobility and strength to manipulate and control these same objects. A common characteristic of the digital articulations is that they are designed to function in flexion. The muscle and ligamentous arrangements compliment this design effectively. Each joint has bilateral collateral ligaments to resist frontal plane motion sagittally. The connective tissue arrangements favor strong flexion as do the more dominant flexor muscles and tendons.

The motion and stability of the fingers are affected by both intrinsic and extrinsic musculature. While the function of each of these categories of muscles is different, it is essential that the resulting forces are coordinated to produce smooth and effective motions for fine manipulation and/or great force.

Hand Kinetics

Research by Von Lanz and Wachsmuth (1970) indicates that the finger flexors are more than twice as strong as the finger extensors. This is due to the inherently larger muscles on the flexor side of the joint as well as to the types of resistances with which these muscles must typically deal. The degree of kinetic activity required in the hand varies with the task. The hand must be capable of providing a strong or powerful grip when dealing with heavy objects or objects that are accelerated at very high rates while being held by the hand (golf clubs, baseball bats, hammers, etc.). Likewise, the hand must be capable of gripping objects under very low loading conditions when control and precision are required or when damage to a delicate object could occur. Additionally, the hand must be capable of relaxing to allow for molding of the palmar surface of the hand and fingers to an object being held. Common tasks for which the hand must accommodate itself include opening a door by grasping and turning the handle; opening a jar; manipulating a pair of scissors or a pencil; gripping a ball, bat, or racquet; pinching an object (tip, palmar, lateral and ulnar pinches); keyboarding; or holding a baby. These varied tasks require great diversity in the forces exerted by, and positioning of, the hand and digits. And the external forces placed on the hand and fingers may be considerable depending upon the type of activity. Some heavy manual labor, sports, and exercise activities can result in very large external forces being applied to the hands thereby causing large reactionary forces. Typically, these reactionary forces are generated by the muscles of the hand and/or fingers. Unfortunately, such reactionary forces can often be imparted to muscles that cannot effectively deal with the force. When these forces are sufficiently large an injury to a ligament, bone or cartilage may occur. Even when the forces are smaller in magnitude, the repetitive nature of the activity may lead to musculoskeletal/neuromuscular problems. Connective tissue disorders (CTDs) are very commonplace in some industries, particularly those involving sewing, data entry (keyboarding), or cutting with a knife (poultry processing plants). Such low force, high repetition activities can lead to muscle fatigue and micro-swelling in the joint space and tendinous sheaths and may lead to problems such as carpal tunnel syndrome or tendinitis.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

The functional exercise gloves disclosed herein provide resistance that induces functional concentric and eccentric contractions of the fingers, hand, wrist, and forearm. In many embodiments, the functional exercise gloves incorporate a flexible, resilient fabric made of, for example, a laminate of natural and/or synthetic rubbers, composites, plastics and textile woven, knitted and or non-woven fabrics. And in many embodiments, the functional exercise glove employes a fabric having assymetrical in-plane resistance to stretch to present resistance in a desired direction while presenting reduced resistance in a direction in which relatively free stretching is desired.

In many embodiments, the ergonomic brace helps to position the hand relative to the forearm in an ergonomically neutral position. The ergonomic braces disclosed in this application can also employ a fabric having assymetrical in-plane resistance to stretch.

This application is related to U.S. Pat. No. 5,453,064, entitled “Exercise Glove Incorporating Rods Which Offer Resistance To Movement of Fingers, Hands, or Wrists,” and to U.S. Pat. No. 5,456,650, entitled “Ergonomic Exercising and Bracing Device,” the entire disclosures of which are hereby incorporated herein by reference. In contrast to the gloves disclosed in U.S. Pat. No. 5,453,064, which can include one or more separate inserted stiffening elements, in many embodiments, the functional exercise gloves disclosed herein employ a composite fabric having an assymetrical resistance to stretch that is built into the dorsal side of the glove. As will be described herein, the length of the composite fabric can be set (shortened or lengthened) to control the level of resistance generated by the glove.

The resistance generated by the glove creates beneficial unbalanced co-contractions in the flexors and extensors in the fingers, hand, wrist and forearm. Such unbalanced co-contractions tend to cause the muscles, tendons, ligaments, and bones to orient the hand and metacarpals (back of the hand closed position) 19 degrees to the radius bone of the forearm, which is anatomically correct. Use of the gloves strengthens the muscles, tendons, ligaments, and bones in the proper anatomical position. And the increased level of muscle contraction serves to teach muscle memory much faster and specific to the movement pattern of the user. This is the result of the new glove and fabric technology that teaches the body to perform a task or function much more rapidly and with precision, because the exercises (concentric and eccentric) are being performed during use. Such exercises are also known as proprioceptive exercises.

In many embodiments, the asymmetric resistance to stretch material in the dorsal side of the glove extends parallel to the back of the hand from the wrist to the ends of the fingers or short of the ends of the fingertips. The asymmetric resistance to stretch material is oriented to align the stiffest direction with the direction of the fingers so as to create increased resistance while allowing the user to perform other functions while wearing the glove. The glove induces isotonic, isometric and isoflexcentric exercises, which are proven to stimulate bone growth, reduce stressors, help to improve performance, and prevent injuries. And the asymmetric resistance to stretch material, as well as other portions of the glove, can incorporate antimicrobials to prevent the spread or growth of microorganisms in the product.

The gloves provide a very effective means of strengthening and training the intrinsic and extrinsic muscles of the fingers, hand, wrist, and forearm for muscular strength and endurance. The construction of the glove provides significant resistance in the sagittal plane of motion. By offering a tensile force on the dorsal surface of the hand and fingers when a grip is attempted from an open/neutral hand/finger position, the glove can challenge the finger flexors to concentrically contract at light to maximum static isometric levels of force. Upon achieving a fully flexed or fist position, the same flexor muscles are challenged eccentrically as the subject slowly returns the fingers to a neutral position. Through continuous finger flexion and slow extension, the intrinsic and extrinsic muscles can be worked concentrically and eccentrically to fatigue. Likewise, the opposing extensor muscles are also activated in an unbalanced co-contraction as they attempt to control the excursions of the flexing and extending bony segments around the various joints of the hand and fingers.

The gloves also provide resistance in the frontal plane of motion. As the user attempts to ab/adduct the fingers from the MCP joint, the glove offers resistance to the involved musculature. This action leads to strengthening of the intrinsic ab/adductors of the fingers.

The exercise gloves also provide tremendous capabilities with regards to working musculature of the lower arm. In addition to basic flexion/extension actions, by balling the grip tightly and circumducting the hand at the wrist, the wrist flexors, extensors, ulnar and radial flexors are all worked as the hand rotates through its full range of motion.

Neuromuscularly, the gloves allow for a variety of activities requiring various degrees of coordination. The glove exercises are proprioceptive in nature, thereby training the muscles rapidly. Since the glove is worn on the hand, the subject can use the glove to grasp other external objects of varying weights and sizes. Therefore, the glove can provide an additional source of resistance to the hand/wrist musculature. Additionally, the user can flex and extend the fingers at slow or fast speeds to illicit varying recruitment of slow and fast-twitch muscle fibers. The gloves provide tremendous latitude in matching the specifics required in a variety of activities ranging from normal rehabilitation tasks to occupational/industrial tasks to sporting/exercise tasks. For example, the subject can simulate keyboarding activities to create endurance and dexterity of the digits for typing.

In addition, one variation of the glove can be used in golf instruction to prevent unwanted hyperextension of the wrist prior to contact in the golf swing. The glove (with built-in bracing component) provides moderate resistance to at least 1 finger while stabilizing the wrist with a dorsal 19°+19° degree brace that crosses the wrist joint. This works exceptionally well in improving golf performance. Another potential application of the glove is in providing temporary, non-cumbersome splinting of the wrist and/or fingers for individuals with problems such as mild carpal tunnel syndrome. The comfortable glove can also be worn to prevent excessive flexion of the fingers and wrist as well as excessive hyperextension of the wrist while sleeping. The gloves can be worn during waking hours by individuals with symptoms of carpal tunnel syndrome to prevent excessive hand and finger positions, preferably only when gripping tasks are not required as the glove would offer additional resistance to that action. The composite fabrics naturally resist finger flexion, wrist circumduction, and/or wrist hyperextension.

Proprioceptive Exercise

Proprioception is the sense of the relative position of neighboring parts of the body. Unlike the six exteroceptive senses (sight, taste, smell, touch, hearing, and balance) by which we perceive the outside world, and interoceptive senses, by which we perceive the pain and movement of internal organs, proprioception is a third distinct sensory modality that provides feedback solely on the status of the body internally. It is the sense that indicates whether the body is moving with required effort, as well as where the various parts of the body are located in relation to each other.

By incorporating functional ergonomic exercise and 19°+19° bracing including isotonic and isometric exercises in motion, the use of the gloves and braces disclosed herein tend to constrain the fingers, hand, wrist, and forearm to ergonomically correct movement patterns. Full flexion, closing the fingers and hand, creates concentric contractions while the dorsal composite fabrics challenge the fingers, hand, wrist, and forearm by concentrically/eccentrically contracting the muscles in the fingers, hand, wrist, and forearm during full closure and extension. The musculature affecting the fingers, hand, wrist, and forearm can also be challenged isometrically at full closure.

These innovative exercise and bracing technologies have the potential of revolutionizing the way individuals, athletes, and workers reach superior levels of strength/endurance and performance as never before. The exercises create unbalanced co-contractions in the flexors and extensors and can be used to cause the extensors to be overloaded thus strengthening them during a flexion or wrist circumduction. The functional ergonomic exercises (e.g., during a golf swing) speed up the muscle memory process exponentially compared to no loading. The devices engage the proprioceptive sensors at a much higher level. In contrast, old strength training techniques involved a workout, totally separate from the activity in which the athlete or worker intended to train. The body, however, adapts very specifically to the training stimuli it is required to deal with. The body will perform best at the specific speed, type of contraction, muscle-group usage, and energy-source usage it has become accustomed to in training. Preferably, an athlete will repeat the appropriate movement patterns in a skillful manner many thousands of times during practice, so the nervous system learns to perform the movement correctly every time. With the devices disclosed herein, proprioceptive training takes place while the user is typing, working, playing an instrument, or involved in a sporting activity, which accelerates the effective learning and strengthening process. It develops neuromuscular balance (timing/muscle memory) and stability, which increase performance and prevent injuries, because the resistance exercises are performed specific to the range of motion of the job function, activity or sport. Endurance can be increased significantly. And flexibility, dexterity, range of motion, and durability can be enhanced greatly, as a result of the functional exercises that are inherent in the devices disclosed herein. The bracing devices disclosed herein can physically enable a user to handle stressors, in the workplace or during sporting activities, thereby preventing the user from being injured.

The exercise gloves can be constructed with soft Cabretta leather in the form of a golf or batting type glove on the palm side and can be constructed with a composite fabric having asymmetrical resistance to stretch on the dorsal side, thereby providing resistance to flexion of the fingers, hand, wrist, and forearm. The composite fabric can be incorporated into the dorsal side of the glove, for example, from the wrist to the end of one or more fingers. The glove can have extension for four fingers and the thumb. The composite fabric(s) provides can be used to produce independent resistance to flexion movements of the fingers and/or thumb. The fabric(s) can be constructed to create varying degrees of elasticity and resistance for the fingers, hand, wrist, forearm muscles, tendons, ligaments, bones, and nerves specific to natural movement patterns while playing a sport or on the job. The glove(s) can also help to prevent injury and/or increase performance. The glove(s) can be used in rehab applications such as for Carpal Tunnel Syndrome, tennis elbow, golfer's elbow, pitcher's elbow, as well as in general from golf, tennis, football, and baseball injuries. The gloves are very comfortable to wear. They are very durable, lightweight, and compact and are easily carried in a jacket pocket, pocketbook, or briefcase.

The basic biomechanics of the exercise glove is to provide primary resistance to flexion of the joints within the hand by the intrinsic and extrinsic musculature. When working against the resistance of the glove as in a hand gripping activity (open hand to clenched fist to open hand), the user first undergoes a concentric contraction of the intrinsic and extrinsic musculature of the finger flexors/hand/forearm followed by an eccentric contraction of the same muscles. The glove provides resistance around the carpometacarpal (CMP), metacarpophalangeal (MCP) and the interphalangeal joints (proximal (PIP) and distal (DIP)). The concentric/eccentric work done against the glove's resistance provides considerable fatigue to the user. There is considerable neuromuscular strength and endurance gained in the finger flexor musculature with regular use of the gloves. Additionally, in the course of performing repeated finger flexion/extension exercises with the gloves, the finger extensors and antagonistic muscles are lengthened and shortened, respectively. As a result, significant co-contractions occur resulting in fatigue of these muscles during prolonged exercise. Therefore, strength and endurance gains of the finger extensors may also be realized by using the gloves.

Thus, in one aspect, a functional exercise glove is disclosed that includes a dorsal-side layer and at least one fingertip member. The dorsal-side layer is configured to cover at least a portion of a dorsal-side of a user's hand when worn by the user. And in many embodiments, the dorsal-side layer covers a majority of the dorsal-side of the user's hand. The dorsal-side layer has a first in-plane resistance to stretch in a first direction and a second in-plane resistance to stretch in a second direction transverse to the first direction. The first in-plane resistance to stretch is greater than the second in-plane resistance to stretch. The at least one fingertip member is configured to transfer an extension/flexion force from at least one finger of the user to the dorsal-side layer upon flexion of the at least one finger. The extension/flexion force internal to the dorsal-side layer is aligned with the first direction. The functional exercise glove is configured to react the extension/flexion force from the dorsal-side layer into at least one of the heel of the user's hand or the user's wrist.

In many embodiments, the functional exercise glove includes a palm-side layer that is coupled to the dorsal-side layer. The palm-side layer is configured to cover at least a portion of a palm-side of the user's hand when the glove is worn by the user. In many embodiments, the functional exercise glove further includes at least one finger gusset layer connected between the dorsal-side layer and the palm-side layer to at least partially define at least one finger of the glove. Each of the at least one finger gusset layer includes an extensible material configured to stretch to accommodate different finger sizes. The at least one finger gusset layer can include a suitable extensible material, for example, spandex.

In many embodiments, the functional exercise glove includes an anchoring mechanism configured to couple the dorsal-side layer with the at least one of the heel of the user's hand or the user's wrist so as to facilitate transfer of the extension/flexion force from the dorsal-side layer into the at least one of the heel of the user's hand or the user's wrist. In many embodiments, the anchoring mechanism includes an adjustable strap. For example, the functional exercise glove can include a hook and loop attachment features to secure the adjustable strap in a selected configuration. And in many embodiments, the functional exercise glove can include a first adjustable strap configured to couple the dorsal-side layer with the heel of the user's hand so as to facilitate transfer of a portion of the extension/flexion force from the dorsal-side layer into the heel of the user's hand and a second adjustable strap configured to couple the dorsal-side layer with the user's wrist so as to facilitate transfer of a portion of the extension/flexion force from the dorsal-side layer into the user's wrist.

In many embodiments, the first in-plane resistance to stretch of the dorsal-side layer is significantly greater than the second in-plane resistance to stretch of the dorsal-side layer. For example, the first in-plane resistance to stretch can be at least five, at least ten, or even at least twenty-five times greater than the second-in plane resistance to stretch.

In many embodiments, the functional exercise glove includes a substantially rigid brace member disposed on the dorsal side of the glove and crossing the user's wrist when the glove is worn by the user. The brace member is configured to constrain the user's wrist in an ergonomically correct position. For example, the brace member can be configured to orient the user's hand up at 19 degrees relative to the radius bone in the user's forearm and to orient the user's hand at 19 degrees ulnar deviation relative to the radius bone in the user's forearm. In many embodiments, the functional exercise glove includes a dorsal-side pocket configured to receive the brace member and interface with the brace member so as to constrain the user's wrist in the ergonomically correct position.

In many embodiments, the at least one fingertip member includes a tip-reinforcement layer having a third in-plane resistance to stretch that is greater than the first in-plane resistance to stretch. Each tip-reinforcement layer wraps around a corresponding fingertip of the glove and is connected to the dorsal-side and palm-side layers.

The dorsal-side layer can be configured to extend any suitable length along one or more fingers of the functional exercise glove. For example, in many embodiments the dorsal-side layer is configured to extend continuously approximately from the tip of a finger to at least the base of the wrist on the dorsal side of the glove such that in an initial relaxed orientation the dorsal-side layer biases the finger into an open or a substantially straight position. In many embodiments, the dorsal-side layer extends from the wrist area of the glove to a position short of the end of at least two fingertips of the glove so as to create more eccentric and concentric resistance to flexion of the muscles, tendons, ligaments, bones, and nerves specific to natural movement patterns of the fingers, hand, wrist, and forearm of the user. In many embodiments, the dorsal-side layer is sewn to the dorsal side of the glove and includes finger extensions that extend up at least two fingers of the glove.

The dorsal-side layer can made from any suitable material. For example, the dorsal-side layer can include a composite including reinforcement fibers and an adhesive matrix. And one or more components of the functional exercise glove can include an antimicrobial agent.

In another aspect, an ergonomic wrist brace is disclosed. The wrist brace includes a flexible strap and a substantially rigid brace member. The flexible strap includes a first end portion, a second end portion, and a middle portion disposed between and connecting the first and second end portions. The first end portion is configured to be wrapped and secured around a user's hand. The second end portion is configured to be wrapped and secured around the user's wrist. The middle portion is configured to cross the user's wrist when the wrist brace is worn by the user. The brace member is coupled to the middle portion of the strap. The brace member crosses the user's wrist and is configured to constrain the user's wrist in an ergonomically correct position. For example, the brace member can be configured to orient the user's hand up at 19 degrees relative to the radius bone in the user's forearm and to orient the user's hand at 19 degrees ulnar deviation relative to the radius bone in the user's forearm.

In many embodiments, the wrist brace includes a pocket configured to receive the brace member. The pocket interfaces with the brace member so as to constrain the user's wrist in the ergonomically correct position.

In many embodiments, the wrist brace further includes a functional exercise glove component that includes at least one finger portion. The functional exercise glove component can be attachable to the wrist brace. The glove component includes a dorsal-side layer and at least one fingertip member. The dorsal-side layer is configured to cover at least a portion of a dorsal-side of a user's hand when worn by the user. The dorsal-side layer has a first in-plane resistance to stretch in a first direction and a second in-plane resistance to stretch in a second direction transverse to the first direction. The first in-plane resistance to stretch is greater than the second in-plane resistance to stretch. The at least one fingertip member is configured to transfer an extension/flexion force from at least one finger of the user to the dorsal-side layer upon flexion of the at least one finger. The extension/flexion force internal to the dorsal-side layer is aligned with the first direction. The glove component is configured to react the extension/flexion force from the dorsal-side layer into at least one of the heel of the user's hand or the user's wrist via the flexible strap.

For a fuller understanding of the nature and advantages of the present invention, reference should be made to the ensuing detailed description and accompanying drawings.

### BRIEF DESCRIPTION OF THE DRAWINGS

FIG. 1 shows a perspective view of a functional exercise glove, in accordance with many embodiments.

FIG. 2 shows a dorsal-side view of another functional exercise glove, in accordance with many embodiments.

FIG. 3 shows a palm-side view of the functional exercise glove of FIG. 2.

FIG. 4 shows a plan view of a 19 degree×19 degree bracing member, in accordance with many embodiments.

FIG. 5 shows a side view of the 19 degree×19 degree bracing member of FIG. 4.

FIG. 6 shows a perspective view of a fingertip reinforcement member in an installed configuration, in accordance with many embodiments.

FIG. 7 shows a cross-sectional view of the fingertip reinforcement member of FIG. 6 in the installed configuration.

FIG. 8 shows a plan view of the fingertip reinforcement member of FIG. 6 in an uninstalled flat-pattern configuration.

FIGS. 9 through 11 show cross-sections of a functional exercise during different stages of a hand gripping motion, in accordance with many embodiments.

FIG. 12 shows a dorsal-side view of another functional exercise glove, in accordance with many embodiments.

FIG. 13 shows a palm-side view of the functional exercise glove of FIG. 2.

FIG. 14 shows a dorsal-side view of another functional exercise glove, in accordance with many embodiments.

FIG. 15 shows a dorsal-side view of another functional exercise glove, in accordance with many embodiments.

FIG. 16 shows a perspective view of an ergonomic brace coupled with a user, in accordance with many embodiments.

FIG. 17 shows the ergonomic brace of FIG. 16 in an uninstalled configuration.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details. Furthermore, well-known features may be omitted or simplified in order not to obscure the embodiment being described.

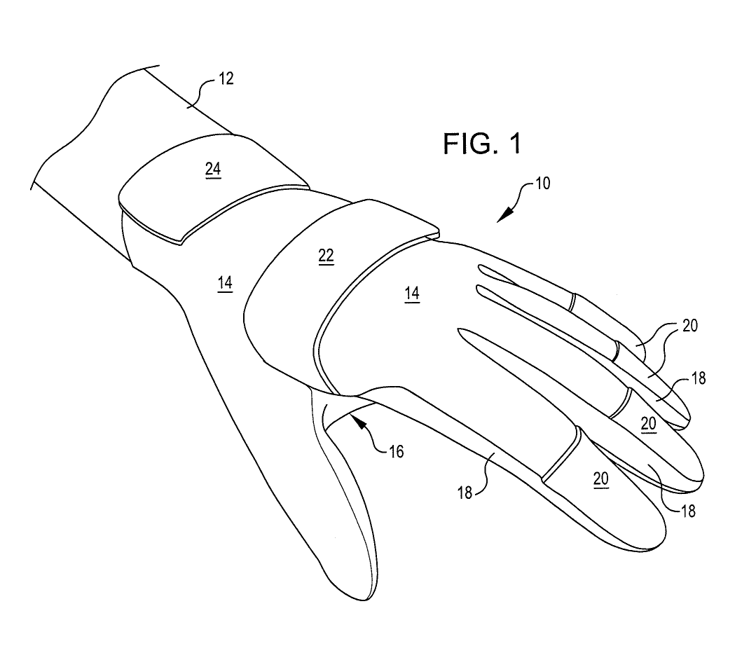

Referring now to the drawings, in which like reference numerals represent like parts throughout the several views, FIG. 1 shows a functional exercise glove 10, in accordance with many embodiments, being worn be a user 12. The glove 10 includes a dorsal-side layer 14, a palm-side layer 16, finger gussets 18, dorsal-side tip portions 20, an adjustable hand strap 22, and adjustable wrist strap 24.

The dorsal-side layer 14 is an engineered fabric that extends from the wrist area of the glove to just short of the fingertips of the glove where the dorsal-side layer 14 is attached to the respective dorsal-side tip portions 20. The dorsal-side layer 14 covers a majority of the dorsal side of the user's hand when the glove 10 is worn by the user 12. The dorsal-side layer 14 has asymmetrical in-plane properties with a first in-plane resistance to stretch in the longitudinal direction of the glove being significantly greater than a second in-plane resistance to stretch transverse to the longitudinal direction. In the glove 10, the longitudinal direction is parallel to the elongate direction of the fingers of the gloves. While the first in-plane resistance to stretch can be greater than the second in-plane resistance to stretch by any suitable amount so that the glove provides a desired level of resistance to finger flexion, in many embodiments the first in-plane resistance to stretch is at least five times greater than the second in-plane resistance to stretch. Additionally, in many embodiments the first in-plane resistance to stretch is at least ten times greater than the second in-plane resistance to stretch. And even further, in many embodiments the first in-plane resistance to stretch is at least twenty-five times greater than the second in-plane resistance to stretch. The higher first in-plane resistance to stretch generates resistance to longitudinal extension of the dorsal-side layer 14, thereby providing resistance against flexion of the user's fingers. The lower second in-plane resistance to stretch permits compliance to transverse changes in the size of the user's hand during flexion of the user's fingers.

Any suitable combination of materials can be used for the dorsal-side layer 14. For example, in one presently preferred embodiment, the dorsal-side layer 14 is made from 0.7 mm thick NL-8050, which is a knitted fabric made from polyester (PET) and polyurethane (PU). The dorsal-side layer 14 can also be made from unidirectional fabrics that are non-woven, web-formed, knitted, woven, and/or laid. The dorsal-side layer 14 can be made in any suitable construction that allows the dorsal-side layer 14 to have low stretch in the warp direction (wrist to finger direction) and higher stretch (e.g., 10 to 30%) in the filling direction (hand side to side). Any fibers, filament yarns, and/or combination yarns used in the dorsal-side layer 14 can be made from any suitable textile manmade polymer including, but not limited to, glass, nylon, polyester (PET), polyurethane, polypropylene, polyethylene, aramids (Kevlar), Nomex, and any other polymers that can be extruded and made into low stretch fabrics. The 0.7 mm thick NL-8050 has a first in-plane resistance to stretch is approximately 102 kg/in̂2 and the second in-plane resistance to stretch is approximately 2.4 kg/in̂2.

In conjunction with the dorsal-side layer 14, the palm-side layer 16, the finger gussets 18, and the dorsal-side tip portions 20 form the main exterior shell of the glove 10. The dorsal-side tip portions 20 form the dorsal-side tip portion of the exterior shell. The palm-side layer 16 forms the palm side portion of the exterior shell. The palm-side layer 16 extends from the wrist area of the glove to the fingertips of the glove on the palm side of the glove 10. The finger gussets 18 form sidewalls of the fingers of the exterior shell. The finger gussets 18 are located on both sides of each finger of the glove 10 and are disposed between and connected to the dorsal-side layer 14 and the palm-side layer 16. In many embodiments, the finger gussets 18 are made from an extendible material configured to stretch to accommodate different finger diameters. While any suitable extendible material can be used to make the finger gussets 18, in one presently preferred embodiment the finger gussets 18 are made from spandex.

The adjustable hand strap 22 and the adjustable wrist strap 24 are positioned along the longitudinal length of the glove such that the dorsal-side layer 14 can be selectively anchored the user's hand and the user's wrist, respectively. The adjustable hand strap 22 is disposed between the wrist and the thumb so that the dorsal-side layer 14 can be selectively anchored to the base of the user's hand (the portion of the user's hand between the wrist and the base of the thumb). And the adjustable wrist strap 24 is positioned at the user's wrist so that the dorsal-side layer 14 can be selectively anchored to the user's wrist or to just distal to the user's wrist. While any suitable configuration adjustment mechanism can be used, in one presently preferred embodiment each of the adjustable hand strap 22 and the adjustable wrist strap 24 employ hook and loop attachment features so as to provide convenient operation and variable adjustment.

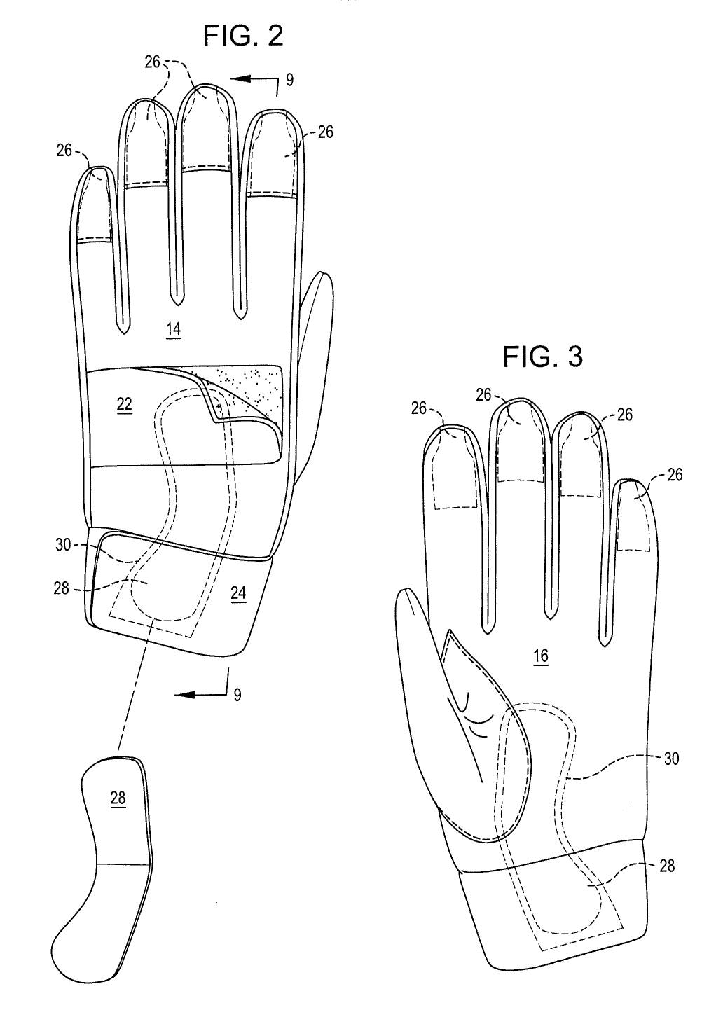

FIGS. 2 and 3 show a dorsal-side plan view and a palm-side plan view of the glove 10, respectively. The glove 10 further includes fingertip members 26 disposed at each of the fingertips of the glove 10. The fingertip members 26 are internal reinforcing members that are made from a relatively in-extendible fabric material. Any suitable material can be used to make fingertip members 26. For example, the fingertip members 26 can be made from nylon and/or polypropylene. And the materials disclosed herein as suitable materials for fabricating the dorsal-side layer 14 can also be used in the fingertip members 26. In many embodiments, the fingertip members 26 have in-plane resistance to stretch parallel to the longitudinal direction of the glove that is greater than the first in-plane resistance to stretch of the dorsal side layer 14. Each fingertip member 26 wraps around the respective fingertip of the glove 10. Each of the fingertip members 26 is connected to the dorsal-side layer 14 and to the palm-side layer 16. Each of the fingertip members 26 is configured to transfer an extension/flexion force from the respective finger of the user to the dorsal-side layer 14 upon flexion of the respective finger. The resulting extension/flexion force internal to the dorsal-side layer is aligned with the longitudinal direction of the glove 10. The increased in-plane resistance to stretch of the dorsal-side layer 14 in the longitudinal direction coupled with the relatively in-extendible fingertip members 26 serve to generate higher level of resistance to flexion of the user's finger as compared to the symmetrical in-plane resistance to stretch of typical glove materials. The fingertip members 26 also serve to reinforce the fingertips of the glove 10, thereby increasing the durability of the fingertips to withstand the increased loading associated with the extension/flexion force that is transferred to the dorsal-side layer 14 upon flexion of the user's fingers.

As also illustrated in FIGS. 2 and 3, the glove 10 can include an optional wrist brace that serves to constrain the user's wrist in an ergonomically correct position. The optional wrist brace includes a substantially rigid brace member 28 and a pocket 30. The pocket 30 is configured to receive the brace member 28 and interface with the brace member 28 so as to constrain the user's wrist in the ergonomically correct position. For example, in many embodiments the brace member 28 is configured to orient the user's hand up at 19 degrees relative to the radius bone in the user's forearm and to orient the user's hand at 19 degrees ulnar deviation relative to the radius bone.

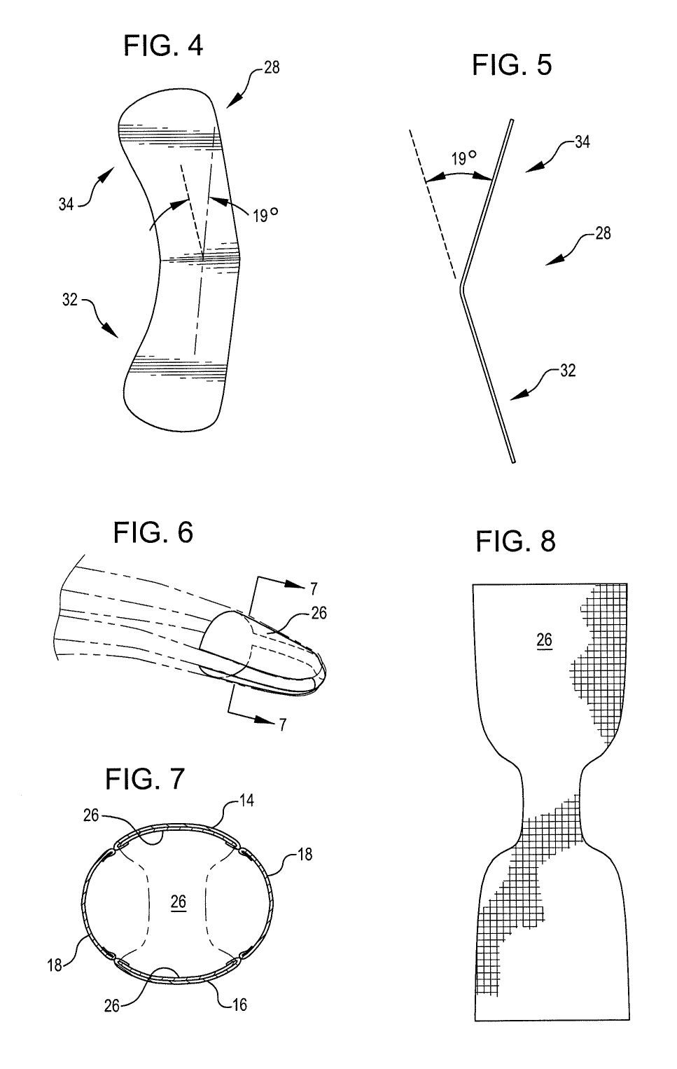

FIGS. 4 and 5 show a plan view and a side view of the brace member 28, respectively. In the embodiment shown, the brace member 28 is made from a constant thickness sheet of material. For example, in many embodiments the brace member 28 has a thickness of 1 mm up to 4 mm depending on the material used. Any suitable material can be used in the brace member 28. Preferably, the material used in the brace member 28 is dimensionally stable and resistant to temperature induced degradation. Example materials that can be used in the brace member 28 include aluminum, stainless steel, urethane, polyester, glass, and fiber reinforced plastic injection molded parts.

The brace member 28 has a configuration with two 19 degree angles. The first 19 degree angle is shown in FIG. 4 in which a centerline of a first end 32 of the brace member 28 is angled relative to a centerline of a second end 34 of the brace member 38 by 19 degrees. The first 19 degree angle serves to orient the user's hand at 19 degrees ulnar deviation relative to the radius bone in the user's forearm. The second 19 degree angle is shown in FIG. 5 in which the second end 34 is angled up relative to the first end 32 by 19 degrees. The second 19 degree angle serves to orient the user's hand up at 19 degrees relative to the radius bone in the user's forearm.

FIGS. 6, 7, and 8 illustrate details of the fingertip members 26. FIG. 6 shows one of the fingertip members 26 in an installed configuration in which the fingertip member 26 is wrapped around a fingertip of the glove 10. FIG. 7 shows cross-section 6-6, which illustrates how the fingertip member 26 is positioned relative to the dorsal-side layer 14, the palm-side layer 16, and the finger gussets 18. FIG. 8 shows a flat pattern of the fingertip member 26.

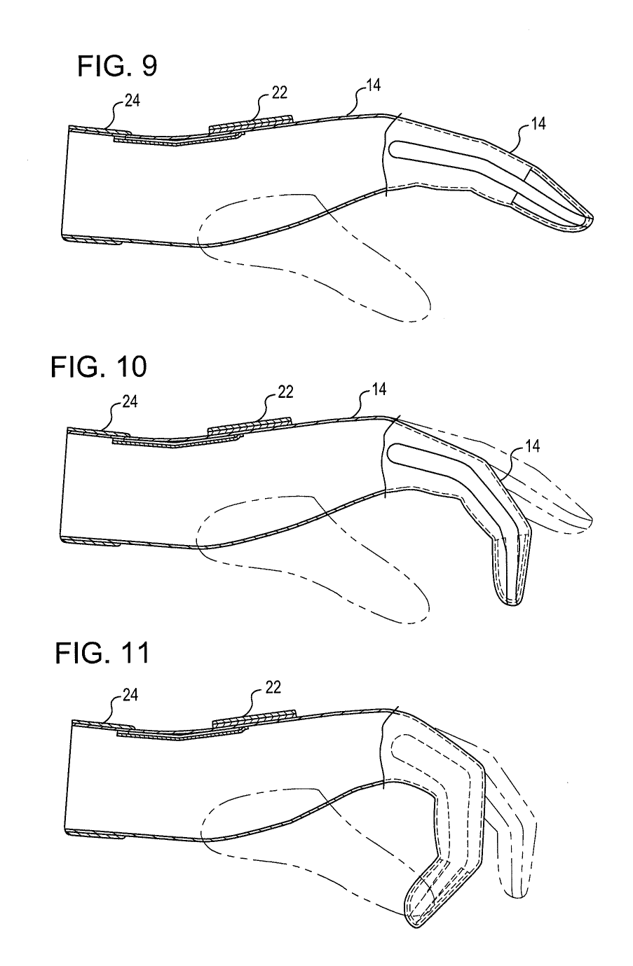

FIGS. 9, 10, and 11 illustrate example positions of a user's hand and the glove 10 during flexion of one of the user's fingers. In FIG. 9, the user's hand is shown in a relaxed state in which the user's fingers are in an extended position. As the user's finger undergoes flexion from the extended position shown in FIG. 9 to the partially flexed position shown in FIG. 10, and further flexion to the flexed position shown in FIG. 11, the length along the dorsal side of the finger is increased. The increased length along the dorsal side of the finger is due to the offset from the dorsal side of the finger to the finger joints. As a result of the increased length along the dorsal side of the finger, the dorsal side of the glove is stretched by an extension/flexion force that is transferred from the user's finger into the dorsal-side layer 14. And as a result of the increased in-plane resistance to stretch of the dorsal-side layer 14 in the longitudinal direction of the glove 10, the extension/flexion force that is required to stretch the dorsal-side layer 14 can be set to a desired level so as to require desired levels of exertion in related muscles of the user. The extension/flexion force internal to the dorsal-side layer 14 is then reacted into the user's hand via the anchoring action of the adjustable hand strap 22 and/or into the user's wrist via the anchoring action of the adjustable wrist strap 24.

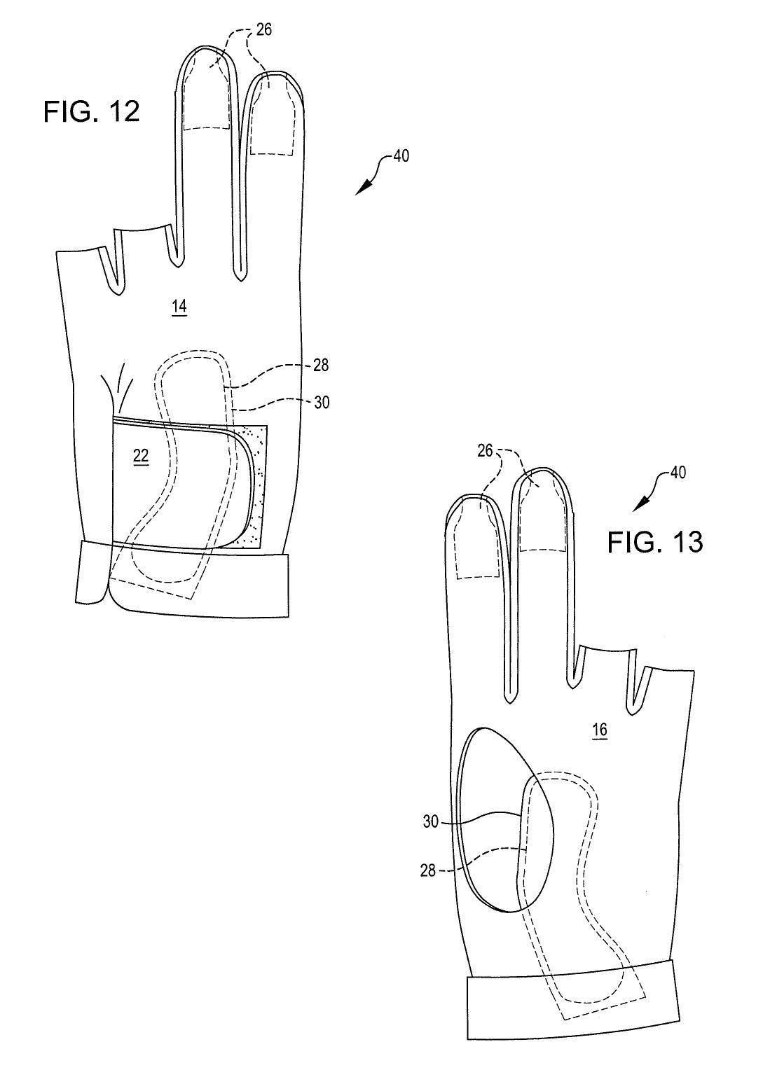

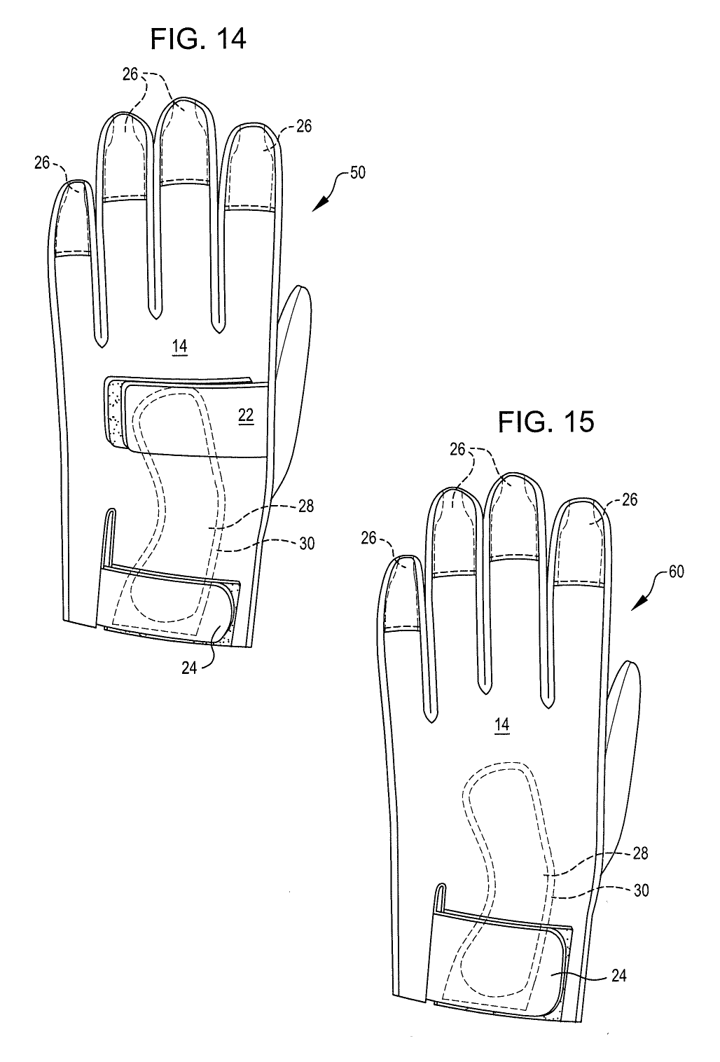

FIGS. 12, 13, 14, and 15 illustrate additional embodiments of a functional exercise glove. FIGS. 12 and 13 show a dorsal-side view and a palm-side view, respectively, of a functional exercise glove 40 that includes two full fingers, an adjustable hand strap 22, and an optional wrist brace. FIG. 14 shows a dorsal-side view of a functional exercise glove 50 that is similar to the glove 10, but includes an adjustable wrist strap 24 having a different configuration. And FIG. 15 shows a dorsal side view of a functional exercise glove 60 with an adjustable wrist strap 24 but no adjustable hand strap.

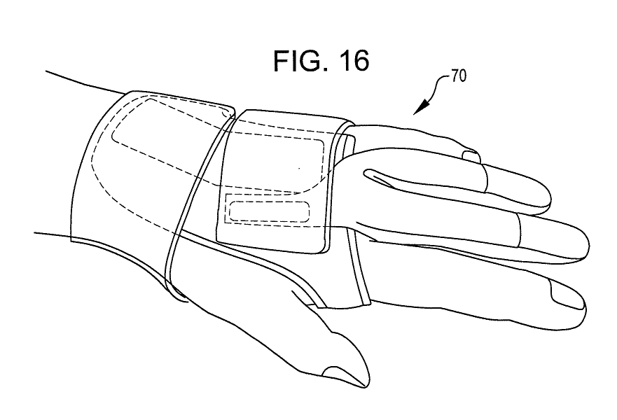

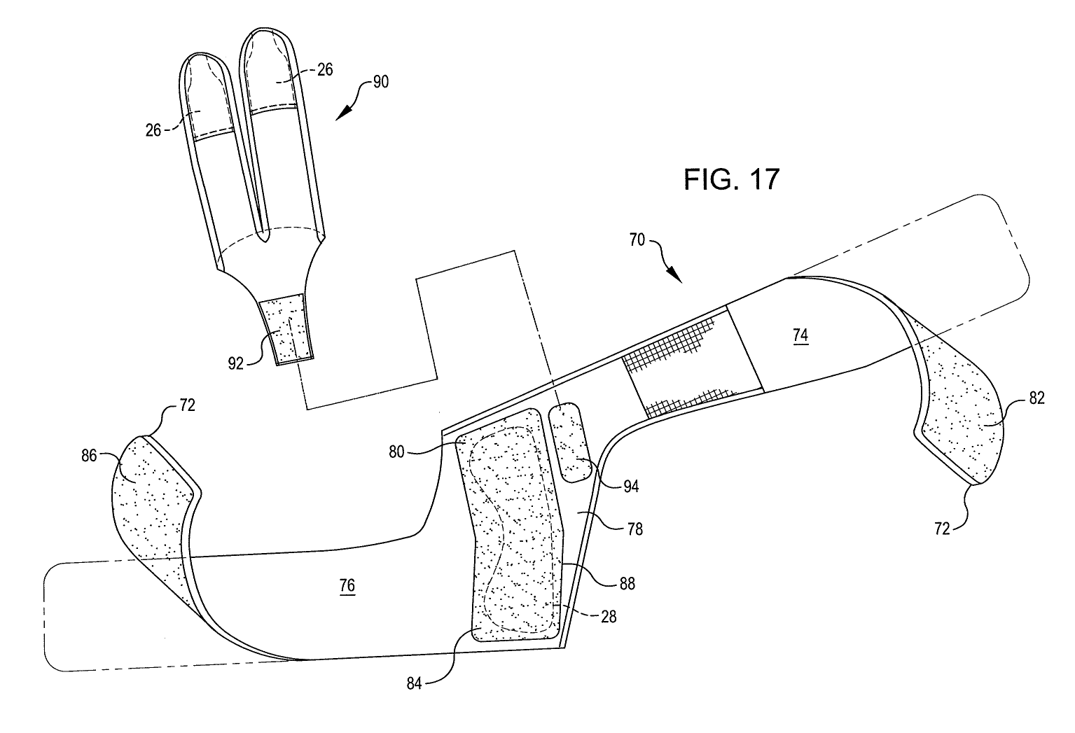

FIG. 16 shows a perspective view of an ergonomic wrist brace 70 coupled with a user, in accordance with many embodiments. FIG. 17 shows the wrist brace 70 when not coupled to a user. The wrist brace 70 includes a flexible strap 72 and a substantially rigid brace member 28 similar to the rigid brace member 28 in the optional wrist brace of the glove 10. The flexible strap 72 includes a first end portion 74, a second end portion 76, and a middle portion 78. The first end portion 74 is configured to be wrapped and secured around a user's hand excluding the user's thumb as shown in FIG. 16. The first end portion 74 includes hook and loop attachment features 80, 82 that engage to secure the first end portion 74 around the user's hand. The second end portion 76 is configured to be wrapped and secured around the user's wrist. The second end portion 76 also includes hook and loop attachment features 84, 86 that engage to secure the second end portion 76 around the user's wrist. The middle portion 78 is disposed between and connects the first and second end portions 74, 76. The middle portion 78 is configured to cross the user's wrist when the wrist brace 70 is worn by the user. The wrist brace 70 includes a pocket 88 attached to the middle portion 78. The pocket 88 is configured to receive the brace member 28 and interface with the brace member 28 so as to constrain the user's wrist in an ergonomically correct position. For example, in many embodiments the brace member 28 is configured to orient the user's hand up at 19 degrees relative to the radius bone in the user's forearm and to orient the user's hand at 19 degrees ulnar deviation relative to the radius bone in the user's forearm.

The wrist brace 70 can include a removable functional exercise glove component 90. While the glove component 90 illustrated has two finger portions, any suitable number of finger portions one or greater can be employed. The glove component 90 and the wrist brace 70 include hook and loop attachment features 92, 94 by which the glove component 90 can be mounted to and demounted from the wrist brace 70. In many embodiments, the glove component 90 includes a dorsal-side layer 14 and fingertip members 26. The dorsal-side layer 14 and the fingertip members 26 of the glove component 90 are configured similar to the dorsal-side layer 14 and the fingertip members 26 of the functional exercise glove 10, respectively. Accordingly, the description relating to the dorsal-side layer 14 and the fingertip members 26 of the functional exercise glove 10 apply to these components of the glove component 90 and will therefore not be repeated here. Similar to the exercise glove 10, the glove component 90 is configured to react the extension/flexion force internal to the dorsal-side layer (imparted into the dorsal-side layer 14 by the user's fingers as a result of flexion of the user's fingers) into at least one of the heel of the user's hand or the user's wrist via the flexible strap 72.

Other variations are within the spirit of the present invention. For example, a user can wear the glove 10 and the wrist brace 70 at the same time by wearing the glove 10 over the wrist brace 70. Thus, while the invention is susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions, and equivalents falling within the spirit and scope of the invention, as defined in the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A functional exercise glove comprising:
a dorsal-side layer configured to cover at least a portion of a dorsal-side of a user's hand when worn by the user, the dorsal-side layer having a first in-plane resistance to stretch in a first direction and a second in-plane resistance to stretch in a second direction transverse to the first direction, the first in-plane resistance to stretch being greater than the second in-plane resistance to stretch; and
at least one fingertip member configured to transfer an extension/flexion force from at least one finger of the user to the dorsal-side layer upon flexion of the at least one finger, the extension/flexion force internal to the dorsal-side layer being aligned with the first direction,
wherein the glove is configured to react the extension/flexion force from the dorsal-side layer into at least one of the heel of the user's hand or the user's wrist.

2. The functional exercise glove of claim 1, further comprising a palm-side layer coupled with the dorsal-side layer and configured to cover at least a portion of a palm-side of the user's hand when the glove is worn by the user.

3. The functional exercise glove of claim 2, further comprising at least one finger gusset layer connected between the dorsal-side layer and the palm-side layer to at least partially define at least one finger of the glove, each of the at least one finger gusset layer comprising an extendible material configured to stretch to accommodate different finger sizes.

4. The functional exercise glove of claim 3, wherein each of the at least one finger gusset layer comprises spandex.

5. The functional exercise glove of claim 1, further comprising an anchoring mechanism configured to couple the dorsal-side layer with the at least one of the heel of the user's hand or the user's wrist so as to facilitate transfer of the extension/flexion force from the dorsal-side layer into the at least one of the heel of the user's hand or the user's wrist.

6. The functional exercise glove of claim 5, wherein the anchoring mechanism comprises an adjustable strap having a strap in-plane resistance to stretch that is greater than second in-plane resistance to stretch.

7. The functional exercise glove of claim 1, further comprising:
a first adjustable strap configured to couple the dorsal-side layer with the heel of the user's hand so as to facilitate transfer of a portion of the extension/flexion force from the dorsal-side layer into the heel of the user's hand; and
a second adjustable strap configured to couple the dorsal-side layer with the user's wrist so as to facilitate transfer of a portion of the extension/flexion force from the dorsal-side layer into the user's wrist.

8. The functional exercise glove of claim 1, wherein the first in-plane resistance to stretch is at least five times the second in-plane resistance to stretch.

9. The functional exercise glove of claim 8, wherein the first in-plane resistance to stretch is at least ten times the second in-plane resistance to stretch.

10. The functional exercise glove of claim 9, wherein the first in-plane resistance to stretch is at least twenty times the second in-plane resistance to stretch.

11. The functional exercise glove of claim 1, further comprising a substantially rigid brace member disposed on the dorsal-side of the glove and crossing the user's wrist when the glove is worn by the user, the brace member being configured to constrain the user's wrist in an ergonomically correct position.

12. The functional exercise glove of claim 11, wherein the brace member is configured to:
orient the user's hand up at 19 degrees relative to the radius bone in the user's forearm; and
orient the user's hand at 19 degrees ulnar deviation relative to the radius bone in the user's forearm.

13. The functional exercise glove of claim 11, further comprising a dorsal-side pocket configured to receive the brace member and interface with the brace member so as to constrain the user's wrist in the ergonomically correct position.

14. The functional exercise glove of claim 2, wherein the at least one fingertip member comprises a tip-reinforcement layer having a third in-plane resistance to stretch that is greater than the first in-plane resistance to stretch, each tip-reinforcement layer wrapping around a corresponding fingertip of the glove and being connected to the dorsal-side and palm-side layers.

15. The functional exercise glove of claim 1, wherein the dorsal-side layer is configured to extend continuously approximately from the tip of a finger to at least the base of the wrist on the dorsal side of the glove such that in an initial relaxed orientation the dorsal-side layer biases the finger into an open or a substantially straight position.

16. The functional exercise glove of claim 1, wherein the dorsal-side layer comprises a composite including an adhesive matrix and at least one of reinforcement fibers, fabrics, an elastomer, or a polymer.

17. The functional exercise glove of claim 1, wherein the dorsal-side layer extends up at least two fingers of the glove from approximately the distal end of the fingers down to approximately the wrist area of said glove.

18. The functional exercise glove of claim 1, wherein the dorsal-side layer extends from the wrist area of the glove to a position short of the end of at least two fingertips of the glove so as to create more eccentric and concentric resistance to flexion of muscles, tendons, ligaments, bones, and nerves specific to the natural movement patterns of the fingers, hand, wrist and forearm of the user.

19. The functional exercise glove of claim 1, wherein the dorsal-side layer is sewn to the dorsal side of the glove and includes finger extensions that extend up at least two fingers of the glove.

20. The functional exercise glove of claim 1, further comprising an antimicrobial agent.

21. An ergonomic wrist brace comprising:
a flexible strap including a first end portion configured to be wrapped and secured around a user's hand, a second end portion configured to be wrapped and secured around the user's wrist, and a middle portion disposed between and connecting the first and second end portions, the middle portion being configured to cross the user's wrist when the wrist brace is worn by the user; and
a substantially rigid brace member coupled to the middle portion, the brace member crossing the user's wrist and configured to constrain the user's wrist in an ergonomically correct position.

22. The ergonomic wrist brace of claim 21, wherein the brace member is configured to:
orient the user's hand up at 19 degrees relative to the radius bone in the user's forearm; and
orient the user's hand at 19 degrees ulnar deviation relative to the radius bone in the user's forearm.

23. The ergonomic wrist brace of claim 21, further comprising a pocket configured to receive the brace member and interface with the brace member so as to constrain the user's wrist in the ergonomically correct position.

24. The ergonomic wrist brace of claim 21, further comprising a functional exercise glove component comprising at least one finger portion, the functional exercise glove component being attachable to the ergonomic wrist brace and comprising:
a dorsal-side layer configured to cover at least a portion of a dorsal-side of a user's hand when worn by the user, the dorsal-side layer having a first in-plane resistance to stretch in a first direction and a second in-plane resistance to stretch in a second direction transverse to the first direction, the first in-plane resistance to stretch being greater than the second in-plane resistance to stretch; and
at least one fingertip member configured to transfer an extension/flexion force from at least one finger of the user to the dorsal-side layer upon flexion of the at least one finger, the extension/flexion force internal to the dorsal-side layer being aligned with the first direction,
wherein the glove component is configured to react the extension/flexion force from the dorsal-side layer into at least one of the heel of the user's hand or the user's wrist via the flexible strap.
